(19) United States
(12) Patent Application Publication
(10) Pub. No.: US 2007/0042987 A1
(43) Pub. Date: Feb. 22, 2007

(54) Stereoselective synthesis of beta-nucleosides

(76) Inventors: Ko-Chung Lin, Lexington, MA (US); Wensen Li, Holmdale, NJ (US); Chunhui Lin, Changhua County (TW); Wein Yungshun, Miaoti County (TW); Kao Kuo-His, Taipei County (TW)

Correspondence Address: FISH & RICHARDSON PC, P.O. BOX 1022, MINNEAPOLIS, MN, 55440-1022, US

(21) Appl. No.: 11/416,380

(22) Filed: May 1, 2006

Related U.S. Application Data
(63) Continuation of application No. 11/194,065, filed on Jul. 29, 2005, now abandoned.
(60) Provisional application No. 60/592,412, filed on Jul. 30, 2004.

(51) Int. Cl. A61K 31/7076; A61K 31/7072; C07H 19/16; C07H 19/19; C07H 19/048; C07H 19/12

(52) U.S. Cl. 514/45; 514/49; 536/28.1; 536/28.3; 536/27.3; 536/27.4; 536/55.3

(57) ABSTRACT

This invention relates to a process of stereoselectively synthesizing an alcohol of the following formula: 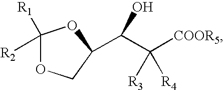
wherein R₁, R₂, R₃, R₄, and R₅ are defined in the specification. The process includes reacting (R) 4-formyl-2,2-dimethyldioxolane with α-bromoacetate in the presence of Zn and a Zn activating agent.

### RELATED APPLICATIONS

This application is a continuation of U.S. patent application Ser. No. 11/194,065, filed Jul. 29, 2005, which claims priority to U.S. provisional application No. 60/592,412, filed Jul. 30, 2004, the contents of which are incorporated herein by reference.

### BACKGROUND

2′-Deoxynucleosides and their analogues are therapeutically important agents. For example, 2′-deoxy-2,2′-difluorocytidine hydrochloride can be used to treat viral infection and cancer (see, e.g., U.S. Pat. Nos. 4,526,988 and 4,808,614).

In general, 2′-deoxynucleosides each have more than one chiral center and can occur as multiple stereoisomers. Not all stereoisomers are therapeutically active. Several stereoselective synthetic routes for 2-deoxy-β-nucleosides have been developed. However, none of them are satisfactory. There is a need to develop a more effective route for stereoselectively synthesizing 2′-deoxynucleosides.

### SUMMARY

This invention is based on an unexpected finding that (R) 4-formyl-2,2-dimethyldioxolane reacts with α-bromoacetate in the presence of Zn and a Zn activating agent (e.g., I₂) to give a 3(R)-hydroxy compound with high enantiomeric purity, i.e., an enantiomeric excess of about 98%. The 3(R)-hydroxy compound is an essential starting material for stereoselective synthesis of certain 2′-deoxynucleosides.

Thus, this invention relates to a process of reacting an aldehyde of the following formula: 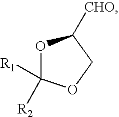
wherein each of R₁ and R₂ independently is H, halo, or alkyl; or R₁ and R₂ together with the carbon atom to which they are attached are a 5 or 6-membered ring; with an ester of the following formula: 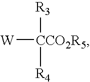
wherein each of R₃ and R₄ independently is H, halo (e.g., F), alkyl, or aryl; R₅ is alkyl or aryl, and W is Br or I; in the presence of Zn and a Zn activating agent (e.g., 1,2-dibromoethane, 1,2-diiodoethane, or I₂) to form an alcohol of the following formula: 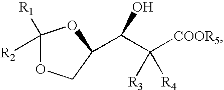
wherein R₁, R₂, R₃, R₄, and R₅ are defined above.

The above reaction can be carried out with microwave, UV, or ultrasound.

To produce a nucleoside, the process includes one or more of the following steps:

(1) transforming the alcohol to a lactone of the following formula: 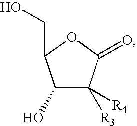
wherein R₃ and R₄ are as defined above;

(2) protecting the hydroxy groups of the lactone to form a protected lactone of the following formula: 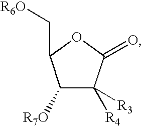
wherein each of R₃ and R₄ are as defined above; and each of R₆ and R₇, independently, is a hydroxy protecting group, or R₆ and R₇, together, are C₁₋₁₃ alkylene;

(3) reducing the protected lactone to a furanose of the following formula: 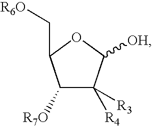
wherein R₃, R₄, R₆, and R₇ are as defined above;

(4) converting the furanose to a furan compound of the following formula: 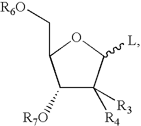
wherein R₃, R₄, R₆, and R₇ are as defined above and L is a leaving group;

(5) reacting the furan compound with a compound of the following formula: 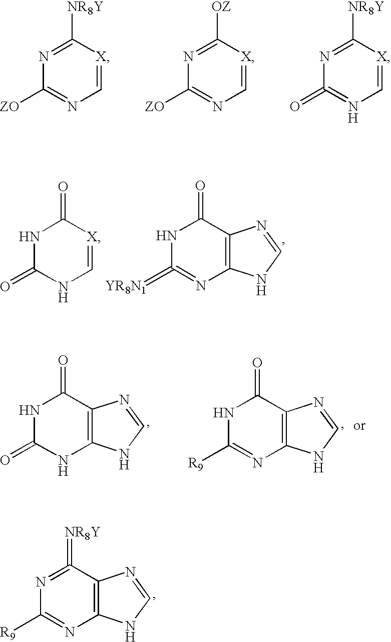
in which R₈ is H, alkyl, or aryl; R₉ is H, alkyl, alkenyl, halo, or aryl; X is N or C—R′, R′ being H, alkyl, alkenyl, halo, or aryl; Y is an amino protecting group, and Z is a hydroxy protecting group; to produce a β-nucleoside compound of the following formula: 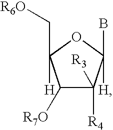
in which R₃, R₄, R₆, and R₇ are as defined above; and B is 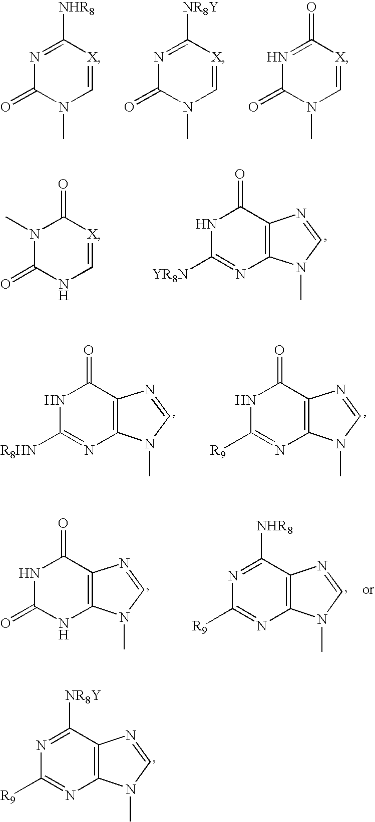
in which R₈ and R₉ are as defined above; and

(8) deprotecting the β-nucleoside to form a 3,5-dihydroxy β-nucleoside of the following formula: 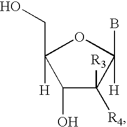
in which R₃, R₄, and B are defined as above.

The term “alkyl” refers to a straight or branched hydrocarbon, containing 1-6 carbon atoms. Examples of alkyl groups include, but are not limited to, methyl, ethyl, n-propyl, i-propyl, n-butyl, i-butyl, and t-butyl. The term “alkoxy” refers to an O-alkyl radical. Examples of alkoxy groups include, but are not limited to, methoxy, ethoxyl, and butoxy. The term “alkylene” refers to a alkyl diradical group. Examples of “alkylene” include, but are not limited to, methylene and ethylene.

The term “alkenyl” refers to a straight or branched hydrocarbon having one or more carbon-carbon double bonds. Examples of alkenyl groups include, but are not limited to, ethenyl, 1-butenyl, and 2-butenyl.

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “alkoxycarbonyl” refers to an alkyl-O-carbonyl radical. Examples of alkoxycarbonyl groups include, but are not limited to, methoxycarbonyl, ethoxycarbonyl, and t-butoxylcarbonyl. The term “aroxycarbonyl” refers to an aryl-O-carbonyl radical.

Examples of aroxycarbonyl groups include, but are not limited to, phenoxycarbonyl and 1-naphthalenoxycarbonyl. The term “aminocarbonyl” refers to a (R)(R′)N-carbonyl radical in which each of R and R′ independently is H, alkyl, or aryl. Examples of aminocarbonyl groups include, but are not limited to, dimethylaminocarbonyl, methylethylaminocarbonyl, and phenylaminocarbonyl.

Alkyl, aryl, alkenyl, and alkoxy mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, and heterocyclyl, in which the alkyl, alkenyl, alkynyl, alkyloxy, aryl, heteroaryl, cyclyl, and heterocyclyl may be further substituted.

The term “furanose” refers to a five-membered cyclic acetal form of a sugar.

Other features, objects, and advantages of the invention will be apparent from the description and the claims.

### DETAILED DESCRIPTION

Referring to Scheme 1, it was unexpectedly discovered that reacting (R) 4-formyl-2,2-dimethyldioxolane 1 with an α-bromoacetate 2 in the presence of Zn and a Zn activating agent (e.g., I₂) gives 3(R)-hydroxy compound 3 with high enantiomeric purity, i.e., enantiomeric excess about 98%.

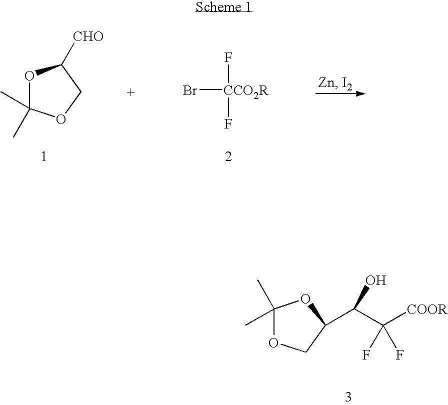

Thus, this invention also features a synthetic process for stereoselectively preparing (R) 3-hydroxy compound 3 and its analogues. The synthetic process includes reacting (R) 4-formyl-2,2-dialkylldioxolane with an alkyl α-Br or α-I substituted acetate in the presence of Zn and a Zn activating agent. The Zn activating agent is a substance that activates Zn metal by reducing any oxidized Zn to atomic Zn. Examples of Zn activating agents include, but are not limited to, I₂, 1,2-dibromoethane, or 1,2-diiodoethane.

The reactants required in this process are commercially available or can be made by methods well known in the art. To practice this process, one can mix the required reactants and a Zn activating agent in a solvent. Examples of suitable solvents include, but are not limited to, dichloromethane, tetrahydrofuran (THF), benzene, chloroform, toluene, xylene, chlorobenzene, hexane, heptane, cyclohexane, hexane, heptane, cyclohexane with ethyl acetate, isopropyl acetate, n-butyl acetate, acetonitrile, 1,2-dichloroethane, and a combination thereof. The Zn activating agent may be employed in a catalytical amount, an equimolar amount, or an excess amount, relative to one of the reactants. The reaction can be carried out at −10 to 30° C. To facilitate this reaction, microwave, UV, or ultrasound can be used. As an example, the reaction vessel can be placed in an ultrasound bath during the reaction. As recognized by those skilled in the art, the reaction time varies depending on the types and the amounts of the reactants, the reaction temperature, and the like.

The product of the above reaction, i.e., 3(R)-hydroxy compound 3, is an important starting material to stereoselectively synthesize certain nucleoside compounds. See, e.g., Chou et al. U.S. Pat. Nos. 4,965,374 and 5,434,254. Scheme 2 below illustrates a synthetic route to 2′-deoxy-2,2′-difluorocytidine from 3(R)-hydroxy compound 3.

Enantiomerically pure 3(R)-hydroxy compound 3 is hydrolyzed to form a lactone 4, namely, 2-deoxy-2,2′-difluoro-1-oxoribose, which is also enantiomerically pure. Lactone 4 has two active hydroxy groups. Before being further reacted, lactone 4 is protected by converting the two hydroxy groups into inactive groups. The protected lactone was then reduced to furanose 5 having a new hydroxy group. The reduction reaction introduces an additional chiral center at the anomeric carbon atom. As a result, 5 furanose 5 is an anomeric mixture. The new hydroxy group of furanose 5 is converted into a leaving group, e.g., methanesulfonate (see compound 6 below), and replaced with cystosine to afford protected 2′-deoxy-2,2′-difluorocytidine. The product is deprotected and purified by column chromatograph to afford the desired β anomer 7.

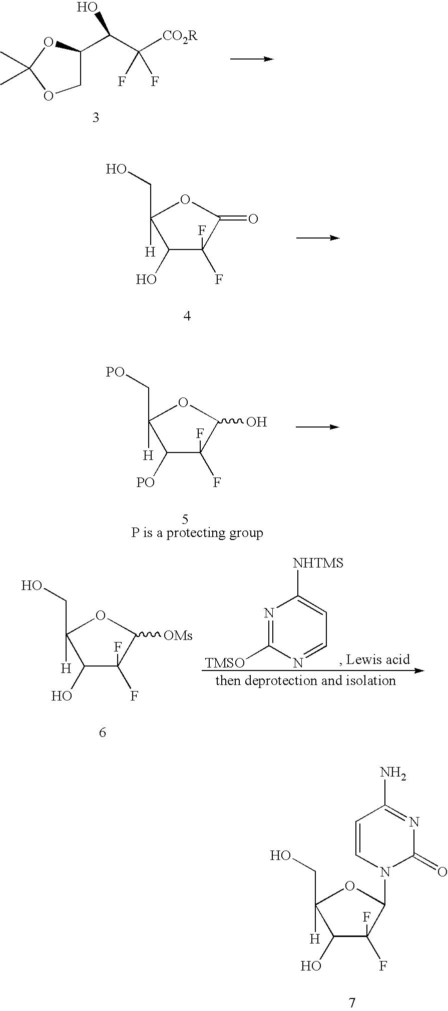

In the above process, several conventional chemical techniques are applied. These techniques include, e.g., introduction of a leaving group, protection and deprotection. A leaving group is a functional group that can depart, upon direct displacement or ionization, with the pair of electrons from one of its covalent bonds (see, e.g., F. A. Carey and R. J. Sundberg, Advanced Organic Chemistry, 3^{rd} Ed. Plenum Press, 1990). Examples of leaving groups include, but are not limited to, methanesulfonate, triflate, p-toluenesulfonate, iodide, bromide, chloride, and trifluoroacetate. Protecting groups refer to those that prevent the protected active groups from interference and can be removed by conventional methods after the reaction. Examples of hydroxy protecting groups include, but are not limited to, alkyl, benzyl, allyl, acyl (e.g., benzoyl, acetyl, or HOOC—X—CO—, X being alkylene, alkenylene, cycloalkylene, or arylene), silyl (e.g., trimethylsilyl, triethylsilyl, and t-butyldimethylsilyl), alkoxylcarbonyl, aminocarbonyl (e.g., dimethylaminocarbonyl, methylethylaminocarbonyl, and phenylaminocarbonyl), alkoxymethyl, benzyloxymethyl, and alkylmercaptomethyl. Examples of amino protecting groups include, but are not limited to, alkyl, acyl, and silyl. Hydroxy and amino protecting groups have been discussed in T. W. Greene and P. G. M. Wuts, Protective Groups in Organic Synthesis, 2d. Ed., John Wiley and Sons (1991).

For the synthetic process described above, completion of the reaction can be monitored by any conventional method, e.g., ultra-violent spectrum, infrared spectrum, nuclear magnetic resonance, thin layer chromatography, gas chromatography, and high performance liquid chromatography. After the reaction is complete, the product can be separated from the reaction mixture by one or more conventional separation methods, such as chromatography, recrystalation, extraction, and distillation. It may be further purified to give higher enantiomeric purity by methods well known in the art. See, e.g., U.S. Pat. No. 5,223,608.

Without further elaboration, it is believed that the above description has adequately enabled the present invention. The following actual example is, therefore, to be construed as merely illustrative, and not limitative of the remainder of the disclosure in any way whatsoever. All of the publications cited herein, including patents, are hereby incorporated by reference in their entirety.

### Preparation of a 2,2-difluoro-3(R)-hydroxy-3-(2,2-dimethyldioxolan-4-yl)propionate

Zn (3.6 g, 57.5 mmol) and 12 (144 mg, 0.6 mmol) was added to a solution of (R)-4-formyl-2,2-dimethyldioxolane (3 g, 23 mmol) and ethyl bromodifluoroacetate (4.7 g, 23 mmol) in THF (50 mL) at 25° C. The reaction vessel was agitated in an ultrasonic bath at 5-10° C. for 12 h. A solution of ethyl bromodifluoroacetate (4.7 g, 23 mmol) in THF (5 mL) was added and the resulting solution was irradiated for additional 12 h at 10° C. The reaction was quenched by a saturated aqueous NH₄Cl solution. The solution was filtered and concentrated in vacuo to ca. 5 mL, diluted with EtOAc (150 mL), washed with brine (15 mL), dried over Na₂SO₄, and concentrated in vacuo to give a crude product. The crude product was purified by flash column chromatography with 10-20% EtOAc-hexane to give a single compound of 2,2-difluoro-3(R)-hydroxy-3-(2,2-dimethyldioxolan-4-yl)propionate (4.4 g, 75% yield) as a yellow liquid.

R_{f}=0.25 in 25% EtOAc-hexane;

¹H NMR (500 MHz, CDCl₃): δ 4.05-4.335 (m, 4H), 4.01-4.04 (m, 2H), 3.29 (br, 1H), 1.32 (t, 3H, J=8 Hz), 1.30 (s, 3H), 1.29 (s, 3H);

¹³C NMR(125 MHz, CDCl₃): δ 163.122 (t, C, J_{C-F}=30.5 Hz), 113.99 (dd, C, J_{C-F}=252 Hz, 254 Hz), 109.70 (C), 73.37 (CH), 71.56(t, CH, J_{C-F}=23 Hz), 65.60 (CH₂), 63.06 (CH₂), 26.09 (CH₃), 24.94 (CH₃), 13.74 (CH₃).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, a 5-membered cyclic compound structurally analogous to the nucleoside compound mentioned above can also be made according to the process of the present invention. Thus, other embodiments are also within the claims.

What is claimed is:

1. A process comprising:
reacting an aldehyde of the following formula: 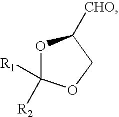
wherein each of R₁ and R₂ independently is H, halo, or alkyl; or R₁ and R₂ together with the carbon atom to which they are attached are a 5 or 6-membered ring; with an ester of the following formula: 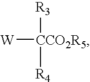
wherein each of R₃ and R₄ independently is H, halo, alkyl, or aryl, R₅ is alkyl or aryl, and W is Br or I; in the presence of Zn and a Zn activating agent to form an alcohol of the following formula: 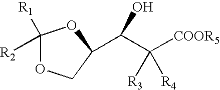
wherein R₁, R₂, R₃, R₄, and R₅ are defined above.

2. The process of claim 1, further comprising:
transforming the alcohol to a lactone of the following formula: 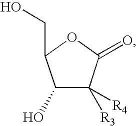
wherein R₃ and R₄ are as defined in claim 1.

3. The process of claim 2, further comprising:
protecting the hydroxy groups of the lactone to form a protected lactone of the following formula: 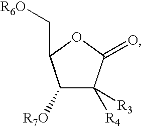
wherein each of R₃ and R₄ are as defined in claim 1; and each of R₆ and R₇, independently, is a hydroxy protecting group, or R₆ and R₇, together, are C₁₋₃ alkylene.

4. The process of claim 3, further comprising
reducing the protected lactone to a furanose of the following formula: 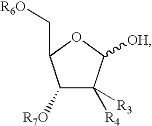
wherein R₃ and R₄ are as defined in claim 1; and R₆ and R₇ are as defined in claim 3.

5. The process of claim 4, further comprising:
transforming the furanose to a furan compound of the following formula: 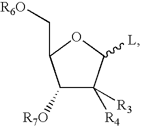
wherein R₃ and R₄ are as defined in claim 1; and R₆ and R₇ are as defined in claim 3; and L is a leaving group.

6. The process of claim 5, further comprising:
reacting the furan compound with a compound of the following formula: 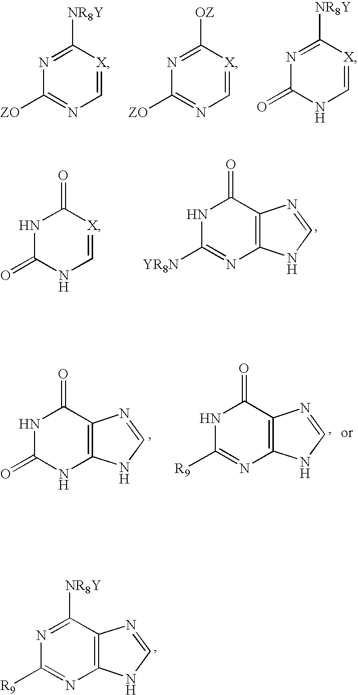
in which R₈ is H, alkyl, or aryl; R₉ is H, alkyl, alkenyl, halo, or aryl; X is N or C—R′, R′ being H, alkyl, alkenyl, halo, or aryl; Y is an amino protecting group; and Z is a hydroxy protecting group;
to produce a β-nucleoside compound of the following formula: 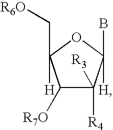
in which R₃ and R₄ are as defined in claim 1; and R₆ and R₇ are as defined in claim 3; and B is 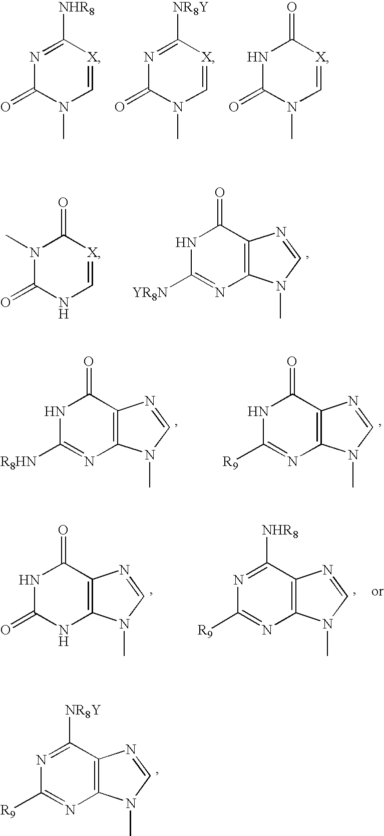
in which R₈ and R₉ are as defined above.

7. The process of claim 6, further comprising:
deprotecting the β-nucleoside to form a 3,5-dihydroxy β-nucleoside of the following formula: 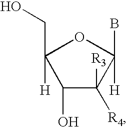
in which R₃ and R₄ are as defined in claim 1; and B is as defined in claim 6.

8. The process of claim 1, wherein the reaction is carried out with microwave, UV, or ultrasound.

9. The process of claim 8, wherein the reaction is carried out with ultrasound.

10. The process of claim 9, wherein the Zn activating agent is 1,2-dibromoethane, 1,2-diiodoethane, or I₂.

11. The process of claim 10, wherein each of R₃ and R₄ is F.

12. The process of claim 11, wherein the Zn activating agent is I₂.

13. The process of claim 9, further comprising:
transforming the alcohol to a lactone of the following formula: 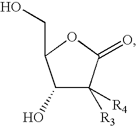
wherein R₃ and R₄ are as defined in claim 1.

14. The process of claim 13, further comprising:
protecting the hydroxy groups of the lactone to form a protected lactone of the following formula: 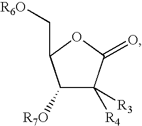
wherein each of R₃ and R₄ are as defined in claim 1; and each of R₆ and R₇, independently, is a hydroxy protecting group, or R₆ and R₇, together, are C₁₋₃ alkylene.

15. The process of claim 14, further comprising
reducing the protected lactone to a furanose of the following formula: 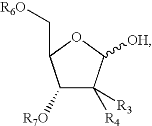
wherein R₃ and R₄ are as defined in claim 1; and R₆ and R₇ are as defined in claim 14.

16. The process of claim 15, further comprising:
transforming the furanose to a furan compound of the following formula: 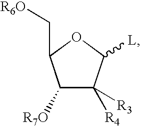
wherein R₃ and R₄ are as defined in claim 1; and R₆ and R₇ are as defined in claim 14; and L is a leaving group.

17. The process of claim 16, further comprising:
reacting the furan compound with a compound of the following formula: 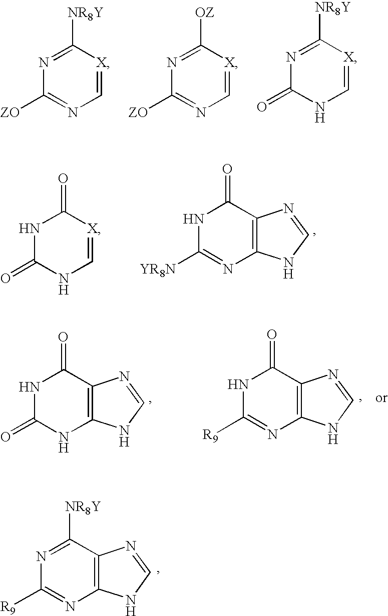
in which R₈ is H, alkyl, or aryl; R₉ is H, alkyl, alkenyl, halo, or aryl; X is N or C—R′, R′ being H, alkyl, alkenyl, halo, or aryl; Y is an amino protecting group; and Z is a hydroxy protecting group;
to produce a β-nucleoside compound of the following formula: 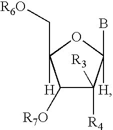
in which R₃ and R₄ are as defined in claim 1; and R₆ and R₇ are as defined in claim 14; and B is 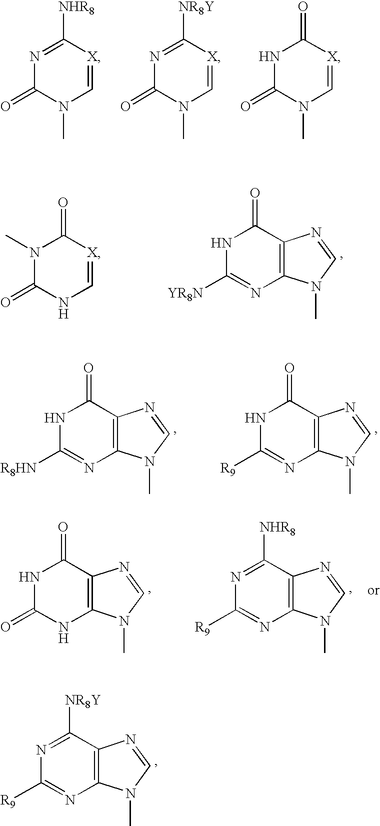
in which R₈ and R₉ are as defined above.

18. The process of claim 17, further comprising:
deprotecting the β-nucleoside to form a 3,5-dihydroxy β-nucleoside of the following formula: 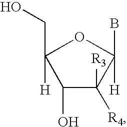
in which R₃ and R₄ are as defined in claim 1; and B is as defined in claim 17.

19. The process of claim 12, further comprising:
transforming the alcohol to a lactone of the following formula: 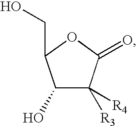
wherein R₃ and R₄ are as defined in claim 1.

20. The process of claim 19, further comprising:
protecting the hydroxy groups of the lactone to form a protected lactone of the following formula: 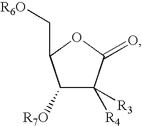
wherein each of R₃ and R₄ are as defined in claim 1; and each of R₆ and R₇, independently, is a hydroxy protecting group, or R₆ and R₇, together, are C₁₋₃ alkylene.

21. The process of claim 20, further comprising
reducing the protected lactone to a furanose of the following formula: 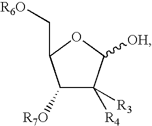
wherein R₃ and R₄ are as defined in claim 1; and R₆ and R₇ are as defined in claim 20.

22. The process of claim 21, further comprising:
transforming the furanose to a furan compound of the following formula: 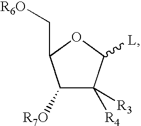
wherein R₃ and R₄ are as defined in claim 1; and R₆ and R₇ are as defined in claim 20; and L is a leaving group.

23. The process of claim 22, further comprising:
reacting the furan compound with a compound of the following formula: 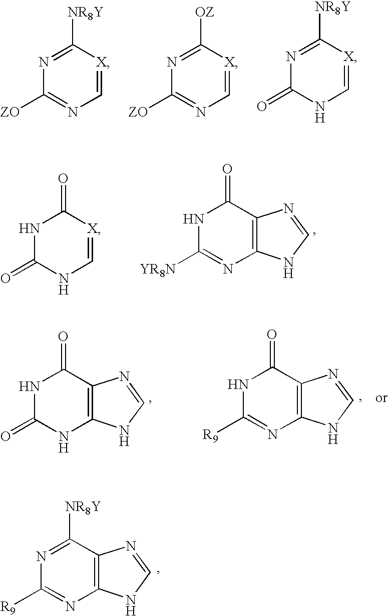
in which R₈ is H, alkyl, or aryl; R₉ is H, alkyl, alkenyl, halo, or aryl; X is N or C—R′, R′ being H, alkyl, alkenyl, halo, or aryl; Y is amino protecting group; and Z is a hydroxy protecting group;
to produce a β-nucleoside compound of the following formula: 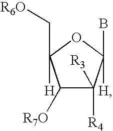
in which R₃ and R₄ are as defined in claim 1; and R₆ and R₇ are as defined in claim 20; and B is 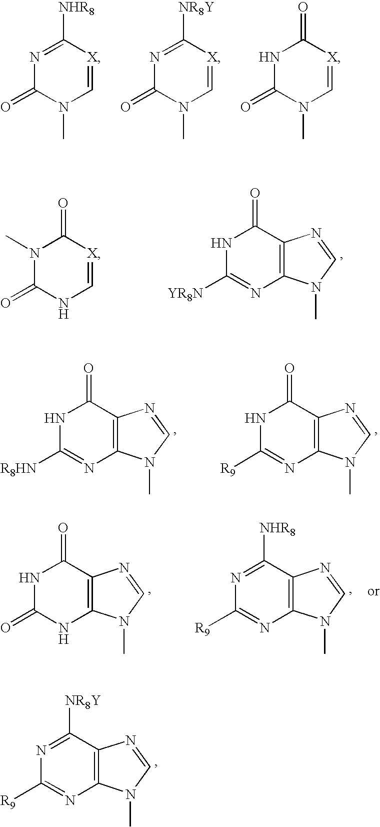
in which R₈ and R₉ are as defined above.

24. The process of claim 23, further comprising:
deprotecting the β-nucleoside to form a 3,5-dihydroxy β-nucleoside of the following formula: 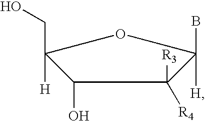
in which R₃ and R₄ are as defined in claim 1; and B is as defined in claim 23.
